(19) United States
(12) Patent Application Publication
(10) Pub. No.: US 2008/0318931 A1
(43) Pub. Date: Dec. 25, 2008

(54) Treatment of Disorders and Diseases of the Colon

(76) Inventors: Moshe Arkin, Kfar Shmaryahu (IL); Irit Gil-Ad, Herzliya (IL); Avraham Weizman, Tel Aviv (IL); Liat Lomnitski, Givat Shmuel (IL); Eilon Asculai, Lehavim (IL); Amira Zeevi, Omer (IL)

Correspondence Address: THE NATH LAW GROUP, 112 South West Street, Alexandria, VA, 22314, US

(21) Appl. No.: 11/631,641

(22) PCT Filed: Jul. 6, 2005
(86) PCT No.: PCT/IL05/00716

§ 371 Date: Mar. 7, 2008

Related U.S. Application Data
(60) Provisional application No. 60/585,840, filed on Jul. 8, 2004.

(51) Int. Cl. A61K 31/55; A61K 31/137; A61K 31/135; A61K 31/343; A61P 1/06; A61K 31/138; A61K 31/4525; A61K 31/381

(52) U.S. Cl. 514/217; 514/656; 514/450; 514/652; 514/647; 514/438; 514/469

(57) ABSTRACT

A method and a composition for the treatment of an Inflammatory Bowel Disease (IBD) or intestine polyposes is described. The method includes the local administration to the colon of a therapeutically effective amount of at least one Selective Serotonin Reuptake Inhibitor (SSRI) or at least one tricyclic antidepressant (TCA).

### FIELD OF THE INVENTION

The present invention concerns drugs for the treatment of diseases of the colon such as inflammatory bowel disease (IBD) by local delivery.

### BACKGROUND OF THE INVENTION

In humans, the majority of inflammatory bowel disease (IBD) occurs in two distinct disorders: Crohn's disease and ulcerative colitis. Both diseases are characterized by chronically relapsing inflammation of the bowel of unknown cause.

Crohn's disease is characterized by a granulomatous, transmural inflammation of the bowel wall, predominantly in the distal ileum. In contrast, ulcerative colitis is defined by crypt abscesses and ulceration limited to mucosa and submucosa and associated with a prominent inflammatory infiltrate.

The mainstay of therapy for IBD is aminosalicylates and topical and systemic glucocorticoids. Sulfasalazine has been used in the treatment of ulcerative colitis for over 55 years. In the colon, the agent is split by bacterial action into sulfapyridine (SP) and mesalamine (5-ASA) which is believed to be the therapeutically active component for the treatment of ulcerative colitis. Both SP and 5-ASA provide therapeutic benefit and improve quality of life to many patients. However, others may still suffer from recurrent disease despite systemic glucocorticoid therapy.

Hydrocortisone and prednisolone are commonly used both in the US and Europe for treating distal colitis. They have clinical efficacy rates ranging from about 60% to 80%. Main concerns associated with their use include systemic side effects and adrenal suppression especially with enema administered steroids that can have as much as 60% bioavailability. (Giller et al., 1980). To minimize these concerns, certain steroid preparations such as those having lower bioavailability have been developed.

Immunosuppressive therapy with therapeutic agents such as cyclosporine and methotrexate have become acceptable for IBD states that are difficult to control with traditional therapy such as glucocorticoids and 5-aminosalicylate analogs. They are frequently used in treating Crohn's disease but less frequent in ulcerative colitis where surgical procedure is the therapy of choice in refractory cases. Some of the side effects of immunosuppressive therapy include bone marrow suppression, nausea and pancreatitis.

The recruitment of leukocytes by cell adhesion molecules, which is induced by interleukin-1 (IL-1), tumor necrosis factor (TNF) and other inflammatory Th1 cells cytokines (such as IL-2 and interferon gamma) mediates the endothelial cell's role in initiating and perpetuating inflammation. Several studies pointed to a necessary mediator function of TNF, particularly in Crohn's disease (Targan et al., 1997; Present et al., 1999). TNF elevation was more pronounced in Crohn's disease than in ulcerative colitis both in plasma (Murch et al., 1991) and mucosal samples (Breese et al., 1994).

Expression of cyclooxygenase-2 (Cox-2), an enzyme which regulates the synthesis of prostaglandins (mediators of inflammation), is strongly increased in epithelial cells, and in mononuclear cells of the lamina propria of Crohn's disease. Over expression of Cox-2 is also observed in the majority of the adenomatous polyps and all adenocarcinomas. Cox-2 inhibitors such as NSAID (celecoxib or rofecoxib) are indicated for the treatment of IBD, as well as for various types of cancer, including colorectal carcinoma and adenomatous polyposis and were reported to be safe and beneficial in most patients with IBD.

Cox-2 was also reported to play a role in the development of several malignancies among which is also the colorectal cancer (Cianchi et al., 2004). In case of colitis, a strong cellular immune response is known from the inflamed regions, i.e. increased presence of neutrophils, neutral killer cells, mast cells and regulatory T cells, which have an important role in the pathophysiology of inflammatory bowel disease.

Cytokine signaling pathways involving transcription factors of the signal transducers and activators of transcription (STAT) family play a key role in the pathogenesis of inflammatory bowel diseases. STAT proteins are latent cytoplasmic transcription factors. STAT-3 levels were significantly increased in IBD patients as compared to controls (Mudter et al., 2005).

Cell proliferation and apoptosis are mediated by activation or inactivation of a variety of genes. Bcl-2 is a protooncogene which exerts an antiapoptotic function, and is overexpressed in states of tumors and cancer including colon cancer. C-jun and its N-terminal kinase (JNK) activate apoptosis (Hu et al., 2005).

Successful treatment with anti-TNF antibodies in patients with Crohn's disease illustrates anti-inflammatory strategies based on the specific blockade of TNF. TNF inhibition by ip injection of the phosphodiesterase inhibitor rolipram is known to attenuate the development of experimental colitis in preclinical tests (in dextran sulfate mouse model).

There are emerging IBD therapies which include biological agents such as infliximab (commercially available as Remicade®) which is expensive and required careful monitoring.

Selective Serotonin Reuptake Inhibitors (SSRIs), such as paroxetine, sertraline, and fluoxetine are the most commonly prescribed antidepressants and are considered as highly effective and relatively safe.

Some SSRIs have shown, in addition to their psychotropic effect, anti-proliferative and anti-inflammatory effects in experimental models. For example, the monocyclic SSRI fluoxetine and zimelidine were shown to inhibit proliferation of prostate carcinoma cells.

PCT/IL01/01105 demonstrates that SSRIs have anti-proliferative effect on several human cancer cells, as well as on non-malignant cells and psoriasis. This application also presents data showing that some psychotropics (phenothiazines and tricyclic antipsychotics) effectively suppress proliferation of human colon carcinoma (HT29 cell line).

The tricyclic antidepressants, TCAs, are a recognized class of structurally related drugs used for the treatment of depression. All of the drugs have an annulated three ring nucleus, a “tricyclic” nucleus, which often has a dibenzazepinyl, dibenzocycloheptadienyl, dibenzoxepinyl, or phenothiazinyl skeleton. A further common feature found in this class of compounds is the presence of a side chain of a substantial length which extends out of one of the atoms, usually carbon or nitrogen, in the central ring of the tricyclic nucleus. The most commonly administered drugs of this class are imipramine, desipramine, amitriptyline, nortriptyline, protriptyline, doxepin, and desmethyldoxepin. Additional discussion of these drug compounds and their analogs' pharmacologic action in treating depression is found in the Physician's Desk Reference (PDR), 58th Ed., 2004, published by Medical Economics Co., Inc., Montvale, N.J., indexed by generic compound name and incorporated by reference herein.

There have been reports concerning the systemic use of some anti-psychotic drugs for the treatment of colitis and Crohn's disease:

Use of 5-HT3 antagonists for the treatment of serotonin-induced gastrointestinal disturbances has been described in U.S. Pat. No. 4,910,193, to Sandoz Ltd.

European Patent No. 0916340 describes use of suppositories containing the active ingredient 6-Amino-5-chloro-1-isopropyl-2-(4-methyl-1-piperazinyl) benzimidazole for treating vomiting accompanying chemotherapy or remedies for irritable bowel syndrome (IBS) based on the serotonin antagonistic activity.

Treating abdominal pain and associated symptoms (constipation and diarrhea) of IBS by paroxetine tablets has been also described. The mechanism proposed for the beneficial effect was the activity of paroxetine at the 5-HT3 receptor or the analgesic effect.

Wang et al. (2003) reported on beneficial treatment of refractory irritable bowel syndrome (IBS) with subclinical oral dosage of antidepressants fluoxetine, paroxetine and doxepin.

US 2004/0014771 discloses the systemic use of combined 5-HT 1A agonists and SSRIs for the treatment of chronic pain as well as for the treatment of IBS.

US 2004/0013741 describes compositions and methods for treating and preventing lower gastrointestinal disorders comprising a gamma-aminobutyric acid analogs, amino-ether or ester oxides in combination with selected GI actives (such as steroids, NSAIDs, antibiotics, 5HT3 receptor antagonists, 5HT3 receptor agonists and SSRIs) provide a more comprehensive reduction in IBS symptoms as compared to previous drug therapies.

The benefit of SSRIs in the irritable bowel syndrome (IBS) has not been clear. In latest randomized trials (Talley, 2004), paroxetine was superior to placebo in terms of improving well-being, but not abdominal pain or bloating. Based on the results of the most recent studies, both tricyclic antidepressants and SSRIs may improve patient satisfaction or quality-of-life without relieving most of the primary gastrointestinal symptoms. This suggests that systemic antidepressant therapy represents only modest efficacy.

In all the mentioned publications, the beneficial effects of psychotropic drugs were related to their known serotonergic effect on GI motility and IBS-associated symptoms (e.g. constipation and diarrhea), but were not associated with their local anti-inflammatory effects.

Furthermore, systemic administration of antipsychotic drugs is associated with many undesirable side effects, among them (for SSRIs): mood swings, headaches, drowsiness, deterioration of sexual functions (sex drive and function) and (for TCAs): drowsiness, anxiety, urinary retention, weight gain, disturbance in sexual drive and function, increase heart beats, etc. Due to those undesirable systemic side effects, the use of SSRIs or TCA for treatment of diseases of the colon has not gained wide acceptance to date.

### SUMMARY OF THE INVENTION

Optimizing the use of antidepressants in IBS and IBD is achieved by local therapy (colon-targeted formulations) instead of immediate release oral administration based on the fact that in the traditional oral route major part of the active is being eliminated by hepatic metabolism (metabolic clearance, first pass effect) and only minimal level of the active drug is distributed into target organs such as colon.

The present invention is based on the finding that several SSRIs such as paroxetine, fluoxetine or sertraline have shown anti-inflammatory effects in human T-lymphocytes and mouse splenocytes. The present invention is further based on the finding that several SSRIs have shown immunomodulating properties mediated by decrease in secretion of cytokines such as IL-2, TNF-γ and IFN-γ. Several TCAs such as clomipramine, doxepine and amitriptyline have also been shown to affect lymphocyte proliferation and splenocyte proliferation. Moreover the SSRIs paroxetine and sertaline were found to suppress the expression of pro-inflammatory enzyme Cox-2 in ConA stimulated mouse splenocytes.

The present invention is further based on the finding that SSRI such as paroxetine and sertaline and some TCAs, process antiproliferative effects on human colon cancel cell line HT27. Notwithstanding these promising results, such SSRI and TCA drugs may be administered locally to the colon in order to decrease to minimum their undesired systemic psychotropic effects. In fact, repeated oral administration of SSRI drugs such as paroxetine to laboratory animals revealed an anti-IBD trend with a dose-response effect.

Thus, the present invention concerns a method for the treatment of an Inflammatory Bowel Disease (IBD) or intestine polyposes in a patient comprising locally administering to the colon a therapeutically effective amount of at least one Selective Serotonin Reuptake Inhibitors (SSRI) or at least one tricyclic antidepressant (TCA).

The intestine polyposes may or may not be associated with any one IBD. The term “intestine polyposes” refers to a lesion/GI proliferative disease (non-malignant tumor) which may be later developed into malignant stage such as colon adenocarcinoma. Such polyposes can also be found in IBD patients who are known to develop significantly higher rates of polyposes and colon carcinoma as compared to normal population. In a preferred embodiment, the method of the present concerns the treatment of such polyposes.

In another embodiment, the method concerns the treatment of inflammatory bowel diseases selected from such diseases as ulcerative colitis, callogenous colitis, microscopic colitis, lymphocytic colitis, ulcerative proctitis, rectocolitis, Crohn's disease, and infectious diarrhea which may or may not be associated with amebiasis, giardiasis, viral infection and pathogenic bacterial infection.

In accordance with the present invention, the active ingredient administered in accordance with the method of the present invention is any SSRI or TCA as known to a person skilled in the art. Preferably, the at least one SSRI is selected from fluoxetine, norfluoxetine, paroxetine, duloxetine, tomoxetine (atomoxetine), nisoxetine, sertraline, desmethylsertraline, fluvoxamine, citalopram, apoxetine and venlafaxine. Most preferably, the SSRI is paroxetine. The preferred at least one TCA is selected from imipramine, clomipramine, amitriptyline and doxepine. Most preferably, the at least one TCA is clomipramine.

In one embodiment, the at least one SSRI or at least one TCA is administered as an oral formulation, preferably in the form of capsules made of polymeric matrices. In one preferred example the polymeric matrix is chitosan.

In yet another embodiment, the at least one SSRI or at least one TCA is administered rectally, preferably using enema or rectal suppository.

The invention further discloses a pharmaceutical composition for the treatment of IBD or intestine polyposes, said composition comprising at least one SSRI or at least one TCA and a pharmaceutically acceptable carrier, excipient or diluent.

In accordance with one embodiment, the IBD is selected from ulcerative colitis, callogenous colitis, microscopic colitis, lymphocytic colitis, ulcerative proctitis, rectocolitis, Crohn's disease, and infectious diarrhea which may or may not be associated with amebiasis, giardiasis, viral infection and pathogenic bacterial infection.

The composition of the present invention comprises any SSRI or TCA as known to a person skilled in the art. Preferably, the at least one SSRI is selected from fluoxetine, norfluoxetine, paroxetine, duloxetine, tomoxetine (atomoxetine), nisoxetine, sertraline, desmethylsertraline, fluvoxamine, citalopram, apoxetine and venlafaxine. Most preferably, the SSRI is paroxetine. The preferred at least one TCA is selected from imipramine, clomipramine, amitriptyline and doxepine. Most preferably, the at least one TCA is clomipramine.

The composition may be formulated in any form and dosage as will be disclosed hereinbelow. Preferably, the composition is formulated for oral or rectal administrations.

Thus, there is also provided an oral composition which comprises at least one SSRI or at least one TCA and pharmaceutically acceptable carrier, excipient or diluent. The active ingredients of the oral composition may be bound or enclosed within a polymeric matrix which is capable of dissociating in the colon, such a matrix carrier may for example be a chitosan capsule.

There is also provided a rectal composition which comprises at least one SSRI or at least one TCA and pharmaceutically acceptable carrier, excipient or diluent. The active ingredients of the rectal composition may be for example dissolved in a rectal solution such as enema or may be embedded or suspended in a rectal suppository.

The invention lastly concerns the use of at least one SSRI or at least one TCA for the manufacture of such a pharmaceutical composition for the treatment of IBD or intestine polyposes.

The term “treatment” or any lingual variation thereof, as used herein refers to administering of a therapeutic amount of the SSRI or TCA used with the present invention which is effective to ameliorate undesired symptoms associated with a disease, to prevent the manifestation of such symptoms before they occur, to slow down the progression of the disease, to slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The expression “Inflammatory Bowel Diseases” or “IBD” refers to such diseases and disorders of the bowel, such as and without being limited thereto, ulcerative colitis, collagenous colitis, microscopic colitis, lymphocytic colitis, ulcerative proctitis, rectocolitis, Crohn's disease and infectious diarrhea such as amebiasis, giardiasis, viral infection or pathogenic bacterial infection (such as E. Coli, Salmonella, Shigella, Campylobacter jejuni and genus Yersinia).

The term “colon” refers to all parts of the colon including the ileum.

The term “colon carcinoma” refers to any colorectal cancer and in particular to adenocarcinoma and squamous cell carcinoma

The term “local administration” or any lingual variation thereof refers to such an administration which ensures that a majority of the active drug remains in the vicinity of the epithelial tissue of the colon either for the treatment of the inflammatory disease (IBD) or the hyperproliferative condition (carcinoma) or for the treatment of polyposes. It should be understood that this term does not mean by any way that none of the drug infiltrates through the colon membranes to the blood circulation (and from there to the CNS), but merely that the amount of systemic effect of the SSRI or TCA drug is minimized so that the undesired side effects, on the central nervous system or the cardiovascular system, are reduced.

The term “therapeutically effective amount” refers to an amount which is sufficient to bring a statistically significant desired effect in one of the diseases as compared to control.

The term “SSRI (Selective serotonin reuptake inhibitors)” refers to drugs which act by selective inhibition of re-uptake of serotonin. Examples of SSRIs include: antidepressant drugs, such as, without being limited thereto, fluoxetine, norfluoxetine, paroxetine, duloxetine, tomoxetine (atomoxetine), nisoxetine, sertraline, desmethylsertraline, fluvoxamine, citalopram, apoxetine and venlafaxine.

The term “TCA (Tricyclic Antidepressants)” refers to antidepressants having a three-cycle structural core. The term refers for example to imipramine, clomipramine, amitriptyline and doxepine.

The active agent according to the invention is dosed in accordance with good medical practice, taking into account the clinical condition of the individual patient, the site and method of administration, scheduling of administration, patient age, sex, body weight and other factors known to medical practitioners.

The compositions of the present invention as well as those used in the methods of the present invention, may also comprise pharmaceutically acceptable carriers such as vehicles, adjuvants, excipients, or diluents, all of which may be well-known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (e) powders; (d) suspensions in an appropriate liquid; and (e)-suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be made of chitosan or of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Rectal formulations are administered through the anal portal into the rectum or intestine. Rectal suppositories, retention enemas or rectal catheters may be used for this purpose as well as any other rectal vehicle which is known to a person skilled in the art. Typically rectal vehicles may be preferred when patient compliance might otherwise be difficult to achieve (e.g., in pediatric and geriatric applications, or when the patient is vomiting or unconscious).

The suppository for example may comprise a central part in which the active ingredient may be comprised. Alternatively, the active ingredient may be in the coating of the suppository or in both the central part or the coating thereof. Additives may be used to advantage in further controlling the desired release rate of the active ingredient for a particular treatment protocol.

In addition to the active ingredients, namely the at least one SSRI or at least one TCA, specific additives such as certain biologically active substances may be included in the suppository or enema. Specific examples of useful biologically active substances include (a) anti-neoplastics such as androgen inhibitors, antimetabolites, cytotoxic agents, immunomodulators; (b) anti-tussives such as dextromethorphan, dextromethorphan hydrobromide, noscapine, carbetapentane citrate, and chlophedianol hydrochloride; (c) antihistamines such as chlorpheniramine maleate, phenindamine tartrate, zyrilamine maleate, doxylamine succinate, and phenyltcloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (l) antacids such as aluminum hydroxide and magnesium hydroxide; (m) biologicals such as peptides, polypeptides, proteins and amino acids, hormones, interferons or cytokines and other bioactive peptidic compounds, such as hGH, tPA, calcitonin, ANF, EPO and insulin; (n) anti-infective agents such as anti-fungals, anti-virals, antiseptics and antibiotics; and (o) antigenic materials, particularly those useful in vaccine applications.

### BRIEF DESCRIPTION OF THE DRAWINGS

In order to understand the invention and to see how it may be carries out in practice, a preferred embodiment will now be described, by way of non-limiting examples only, with reference to accompanying drawings, in which:

FIG. 1 shows the effect of paroxetine, clomipramine and dexamethasone on basal viability and on ConA-induced splenocyte proliferation.

FIG. 2 shows the effect of paroxetine, clomipramine and dexamethasone on ConA-induced mouse splenocyte proliferation and on TH1 cytokines (INF-γ, IL-2 and TNF-α) secretions.

FIG. 3 shows the effect of paroxetine, clomipramine and fluoxetine on thymidine incorporation in mouse splenocytes (basal and ConA treated).

FIG. 4 shows the effect of SSRIs (paroxetine and sertraline) as compared to dexamethasone on ConA-induced stimulation of splenocytes.

FIG. 5 shows the effect of different antidepressants on INF-γ secretion in ConA-stimulated mouse splenocytes.

FIG. 6 shows the effect of different antidepressants in TNF-α secretion in ConA-stimulated mouse splenocytes.

FIG. 7 shows the effect of antidepressants on viability of ConA-stimulated naïve mouse splenocytes.

FIG. 8 shows ConA to basal ratio of splenocytes derived from naïve mice treated with paroxetine or clomipramine.

FIG. 9 shows the effect of paroxetine on PHA-iriduced human lymphocyte proliferation.

FIGS. 10A-B show the effect of psychotropics on viability of human PBMC.

FIG. 11 shows Cox-2 expression in naïve mice splenocytes treated with antidepressants and dexamethasone (normalized to actin).

FIG. 12 shows the effect of dexamethasone, paroxetine and sertraline on viability of ConA activated spleocyte derived from naïve, LPS mice and LPS mice treated with sertraline.

FIG. 13 shows the effect of thioridazine, perhenazine, fluphenazine, clozapine, 5FU and doxorubicin on viability of human colon carcinoma.

FIG. 14 demonstrates the viability of human carcinoma (HT-29) cells treated with psychotopics.

FIGS. 15A-B show the effect of local subcutaneous administration of sertraline on HT-29 human colon cancer tumor growth: FIG. 15A shows the untreated control HT-29 inoculated; FIG. 15B shows the sertraline treated mouse.

### DETAILED DESCRIPTION OF THE INVENTION

### 1. Local Administration to the Colon

The term “local administration” as indicated above refers to a sort of application which is targeted mainly to the colon. This does not mean that some of the active ingredient does not infiltrate to the blood stream, but meaning that most of the drug, and most of the effect, are localized to the endothelial wall of the colon. The local administration may be achieved as follows:

### 1.1 Rectal Administration

Compositions mused for rectal administration, such as by use of an enema, or a suppository composition, are formed with the view that the composition should be maintained in the colon as long as possible in order to minimize absorption to the blood and minimize undesired side effects.

### 1.2 Bio-Targeting to Inflamed Tissue

By this approach, biodegradable particles and/or dextrines are absorbed by activate macrophages in inflamed colon (bio-targeting due to high affinity of this carrier to inflamed colon).

### 1.3 Cleavage of Glucosylated Compounds

Glucosylated products or covalent linkage of drugs with carrier can be administered to the colon utilizing cleavage by colon flora, in a similar manner as oral dosage forms using pro-drug or drugs with active metabolites/active degradation product. Examples are sulfasalazine enema for ulcerative colitis and proctitis which are cleaved by bacterial action in the colon into sulfapyridine (SP) and mesalamine (5-ASA). It is thought that the mesalamine component is in a therapeutically active form in ulcerative colitis. A similar concept can be used also to target other drugs.

The mechanism of action of mesalamine (and sulfasalazine) is unknown, but appears to be topical rather than systemic. Although the pathology of inflammatory bowel disease is uncertain, both prostaglandins and leukotrienes have been implicated as mediators of mucosal injury and inflammation. Recently, however, the role of mesalamine as a free radical scavenger or inhibitor of tumor necrosis factor (INF) has also been postulated.

### 2. Local Treatment of the Colon by Oral Administration

There are several different approaches for ensuring that a drug, administered orally, is mainly targeted to the colon and these are summarized briefly as follows:

2.2 Covalent Linkage of Drugs with Carriers

This approach is achieved by linking the SSRI or TCA and a carrier in such a manner that upon oral administration the drug remains intact in the stomach and small intestine (as a prodrug) and is spontaneously or enzymatically cleaved or released in the colon. For example, the SSRI or TCA may be conjugated via an azo bond which is cleaved by reduction of the azo group by colon bacteria thus releasing the drug.

Another approach is the formation of a glycoside-carrier using the glycosidase activity of colonic microflora to degrade the glycoside-drug conjugate and thus release the carrier.

Chitosan, which is a high molecular weight cationic polysaccharide derived from naturally occurring chitin in crab and shrimp shells was shown to disintegrate by the microorganisms which are abundantly distributed in the colon. Capsules made of chitosan have been found to be generally useful and effective in delivering various drugs to the colon.

Another possibility is to form a conjugate with glucuronide and sulfate with the drug which is released by the de-glucuronidation process in the colon.

Still yet another possibility using carrier is the use of cyclodextrine conjugates, which tend to form inclusion complexes with the drug molecules and which are known to be barely hydrolyzed and slightly absorbed during the passage through the stomach and small intestine, however the cyclodextrine conjugates are known to be fermented by colonic microflora into small saccharides thereby releasing the active ingredient in the colon.

Yet another possibility for carrier is the use of dextran conjugates using a dextran ester pro-drug.

Still yet another possible carrier is an amino acid conjugate which make use of the hydrophilic nature of the polar groups of NH₂ and COOH which reduce the membrane permeability of compounds containing them. Thus, it is possible to produce a prodrug, targeted mainly to the colon by conjugating of the drug to polar amino acids.

### 2.3 Coating by Polymers

The intact SSRI or TCA may be delivered to the colon without absorbance in the upper part of the intestine by coating the drug molecule with suitable polymers which are degradable only in the colon.

pH-sensitive polymers that exploit the change of pH from an acidic pH in the stomach, to a relatively basic pH in the distal ileum may be used. The most commonly used pH dependent coating polymers are meta-acrylic acid copolymers such as Eudragit® S., Eudragit® L. (Rohm Pharmaceuticals, Germany) or which make use of meta-acrylic resin coating.

Another coating approach is coating with biodegradable polymers which make use of the degradation of polymers by the colonic microflora. Typically, these polymers are polymers which make use of the degradation of azo bonds by azo-reductase enzymes produced by azo bacteria present in colonic microflora.

2.4 Embedding in Matrixes

The TCA or SSRI drug molecule may be embedded in a polymeric matrix which shows the ability in the colon for the liberation of the entrapped drug. The embedding may be in a biodegradable matrix or in a biodegradable hydrogel and mainly uses polysaccharides which retain their integrity in the GI, as they are resistant to digestive action of gastrointestinal enzymes, and are active only in the colon upon degradation with bacterial polysaccharidases that result in the degradation of the matrices or polygels. Examples of polysaccharides are amylose, guar gum, pectin, chitosan, insulin, cyclodextrine, chondroitin sulfates, dextrans, etc. As these polysaccharides are typically soluble in water, they are made water insoluble by cross-linking or by hydrophobic derivativization so that the insoluble derivatives of these polysaccharides should be used as the matrix material.

Hydrogels are typically formed by the covalent cross-linking of linear hydrophilic polymers to form a network of material capable of absorbing water yet remaining insoluble. Hydrogel based on the azo polymeric network have been developed for site-specific delivery of drugs to the colon.

### 2.5 Embedding in pH Sensitive Matrixes

This is a similar approach to the binging to pH sensitive polymers, but here the drugs are embedded in a matrix which is relatively resistant to the acidic pH in the stomach and is degraded by the more basic pH in the colon.

### 2.6 Time Release System

Some drug delivery systems are based on the delayed delivery of drugs, typically to 3-4 hours after administration to ensure that do not disintegrate in the stomach or the intestine. This can be achieved for example by use of hard gelatin capsules filed with hydrogel which have long resistance to dissolution, by use of multiple-layers coating (some of which are pH resistant), by use of specifically formulated enteric-coated time release press coated tablets and more.

### 2.7 Redox Sensitive Polymers

Azo or disulfide compound can undergo degradation due to influence of redox-potential in the colon, so that non-cross-linked redox-sensitive polymers containing azo and/or disulfide linkages can be used to embed the drug within.

### 2.8 Bio Adhesive Systems

Bio adhesion is a process by which dosage form remains in contact with particular organs for long periods of time various polymers including polycarbophils, polyretens and polyethyleneoxide, polypropyleneoxide copolymers may be used as bio adhesive systems.

2.9 Coating with Microparticles

There are some new approaches using small silica particles covalently linked to the drug, which can be used to protect the drug from degradation in the stomach.

### 2.10 Osmotic Controlled Drug Delivery

This delivery system may use a single osmotic unit or may have several (up to 6 push-pull units) which are all encapsulated in a single hard gelatin capsule. These capsules having a drug-impermeable enteric coating are prevented from absorbing water in the aqueous environment of the stomach and therefore no drug is delivered there. As the unit of the drug enters the small intestine the coating dissolves in the high pH environment and water enter the unit causing the osmotic push compartment to swell and create a flowable gel in the drug compartment. Swelling of the osmotic “push” compartment forces the drug gel out of the orifice at a rate precisely controlled by the rate of water transferred through a semi-permeable member separating the two. These systems can be precisely tailored to release the active ingredients a specific number of hours, for example, 3-4 hours post gastric delay in order to prevent the drug delivery in the small intestine so that the drug release begins when the unit reaches the colon.

The invention will now be described by way of examples with reference to the accompanying Figures. While the foregoing description describes in detail only a few specific embodiments of the invention, it will be understood by those skilled in the art that the invention is not limited thereto and that other psychotropic agents may be applied to other types of proliferative diseases, without departing from the scope of the invention as defined by the following.

### 3. Pharmaceutical Compositions for Administration for Treatment of IBD

The following Examples contain pharmaceutical compositions in which antidepressants of the TCA or SSRI families were formulated for use in treatment of inflammatory bowel diseases.

Composition 1: Tablet-Composition per core using meth-
acrylic acid copolymer B which are colon-targeted

| | | |
|---|---|---|
| Paroxetine | 100-1000 | mg. |
| Crospovidone | 40-110 | mg. |
| Polyvinyl polypyrrolidone | 20-90 | mg. |
| Polyvinyl pyrrolidone | 20-80 | mg. |
| Magnesium stearate | 10-50 | mg. |
| Methacrylic acid copolymer B | 20-210 | mg |
| Dibasic Calcium Phosphate | | |
| Maltodextrin | | |
| Lactose | | |

Composition 2: Enema composition using methyl
cellulose which is colon targeted

| Ingredient | (% w/w) |
|---|---|
| Paroxetine | 0.1-10 |
| Sodium chloride | 0.15-2.0 |
| Sodium hydroxide | 0.05-1.0 |
| Edetat disodium | 0.05-3.0 |
| Carbopol | 0.02-1.2 |
| Xanthan Gum | 0.1-1.0 |
| Methyl cellulose | 0.1-1.0 |
| Polyoxyethylene sorbitan Monooleate | 0.05-0.5 |
| Ascorbic acid/Sodium benzoate/ | 0.03-0.7 |
| Benzalkonium chloride (50% solution) | 0.03-1.0 |
| Potasium metabisulfite | 0.15-2.0 |
| P. water | q.s to 100% |

Composition 3: Enema composition using methyl
cellulose which is colon targeted

| Ingredient | (% w/w) |
|---|---|
| Paroxetine | 0.1-10 |
| Sodium chloride | 0.15-2.0 |
| Sodium hydroxide | 0.05-1.0 |
| Edetat disodium | 0.05-3.0 |
| Carbopol | 0.02-1.2 |
| Xanthan Gum | 0.1-1.0 |
| Methyl cellulose | 0.1-1.0 |
| Polyoxyethylene sorbitan Monooleate | 0.05-0.5 |
| Ascorbic acid/Sodium benzoate/ | 0.03-0.7 |
| Benzalkonium chloride (50% solution) | 0.03-1.0 |
| Potasium metabisulfite | 0.15-2.0 |
| Methoxy PEG | 20 |
| PEG 400 | q.s to 100% |

Composition 4: Suppository composition using micro-
crystalline cellulose which is colon targeted

| | | |
|---|---|---|
| Paroxetine | 100-1000 | mg |
| Witepsol W-35/Hard fat | 1500-3000 | mg |
| Macrogol 6000 | 300-1000 | mg |
| Microcrystalline cellulose | 300-1000 | mg |
| Magnesium stearate | 2-10 | mg |
| Talc | 2-10 | mg |
| Polyvinyl pyrrolidone | q.s. to 1500-3000 | mg |

Composition 5: Capsule using ethylcellulose which is colon targeted

| | | |
|---|---|---|
| Paroxetine hydrochlorid | 200-1000 | mg |
| Monoglycride acetylated | 10-700 | mg |
| Castor oil | 3-10 | mg |
| Colloidal silicon dioxide | 3-10 | mg |
| Hydroxypropyl methylcellulose | 10-700 | mg |
| Satiric acid | 3-30 | mg |
| Ethylcellulos | 10-700 | mg |
| Talc | 10-700 | mg |
| White wax | 3-10 | mg |

### 4. Experimental Results

### Example 1

### Effect of the Antidepressants Paroxetine (SSRI) and Clomipramine TCA) as Compared to Dexamethasone at Concentrations of 2.5-10 μM on Basal and Concavalin A- (ConA) 5 μM Induced Cell Proliferation

Mouse splenocytes were isolated from C57b1 healthy female mice. Cells (10,000/well) were tested under basal and ConA stimulation. Cells were treated with vehicle, dexamethasone or antidepressants (2.5-10 μM). Viability was assessed 48 hr later using alamar blue staining. (Reagents were manufactured by Wildflower, Santa Fe, N. Mex., USA.) Alamar blue assay was performed as in Nociari et al (1998).

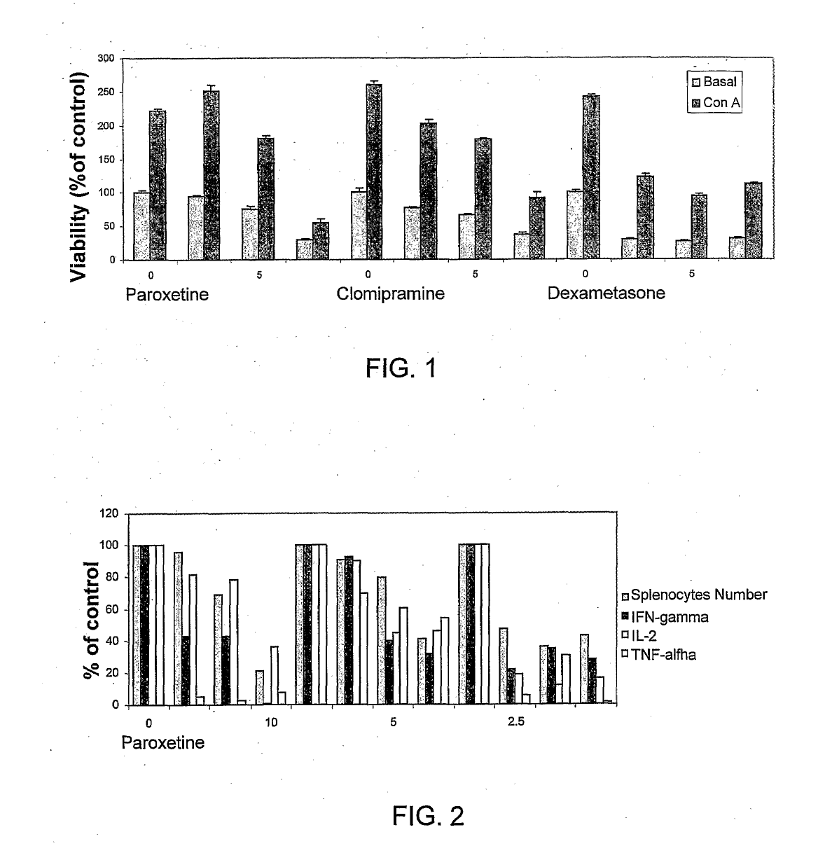

Results were plotted in FIG. 1, wherein each point is the mean of 4 determinations.

The data demonstrates that the SSRI paroxetine and the TCA clomipramine induced a dose dependent decrease in the basal and the mitogen-induced splenocyte proliferation resembling the pattern produced by the corticosteroid dexamethasone (shown for reference only).

### Example 2

### Effects of Paroxetine and Clomipramine on Con-A-Induced Mouse Splenocyte Proliferation and Secretion of IL-2 and INF-γ

Effect of paroxetine, (SSRI) and clomipramine (TCA) as compared to dexamethasone on Con-A-induced splenocyte proliferation (alamar blue method), and splenocyte-induced secretion of TH1 cytokines TNF-α, IL2 and INFγ, 1×10⁶ cells/well, was measured 48 hours after exposure to drugs (2.5-10 μM).

Cytokines were determined in the supernatant of the splenocyte specimens using an ELISA kit and protocol (Reagents by Cytolab). As may be concluded from FIG. 2, paroxetine (SSRI) and clomipramine (TCA) caused a dose-dependent decrease in splenocyte proliferation, which led to a decrease in the level of lymphocyte-induced pro-inflammatory cytokine secretion. The pattern observed resembles that of dexamethasone. Thus, the results indicate that SSRI and the TCA agents produce glucocorticosteroid-like anti-inflammatory effects.

### Example 3

### Effect of the SSRIs Paroxetine and Fluoxetine and TCA Clomipramine on Basal and ConA-Induced Splenocytes Proliferation as Measured by Thymidine Incorporation

Mouse splenocytes were isolated from C57b1 healthy female mice. Cells (10,000/well) were tested under basal and ConA stimulation. Cells were treated with vehicle, or antidepressants (2.5-10 μM). ³H-thymidine, 1 μCi/ml, was added to the cells for 24 hr, after which period the cells were harvested and the incorporate radioactivity was determined by a liquid scintillation beta counter.

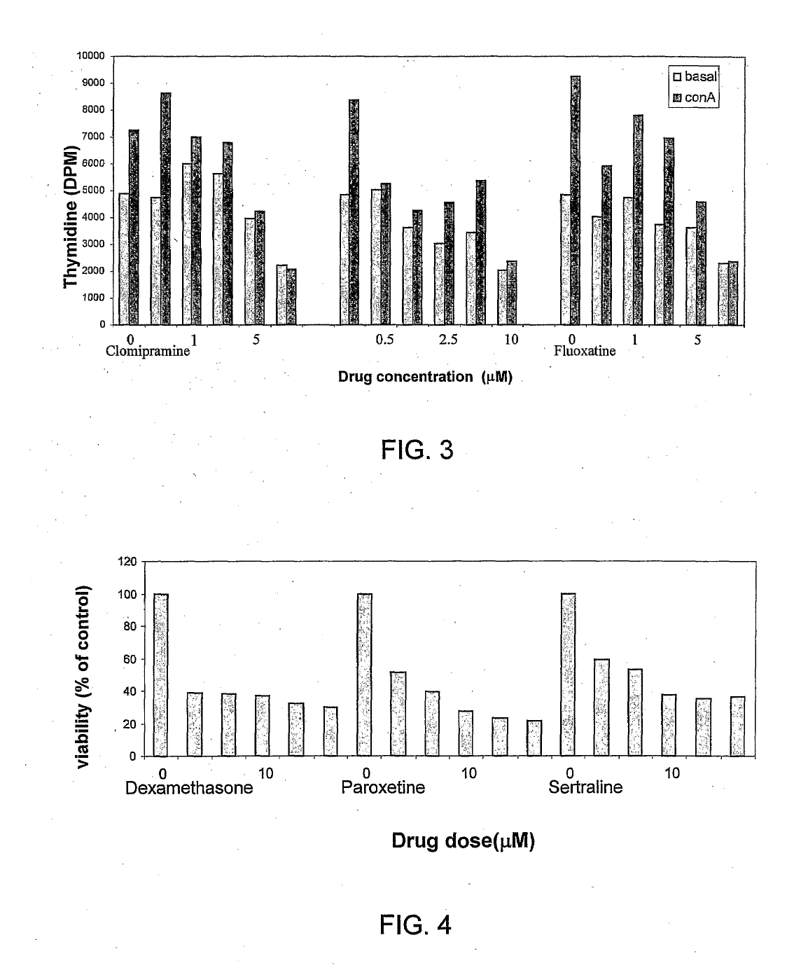

Each point on the graph is the mean of 4 determinations. The results shown in FIG. 3 indicate that all 3 antidepressants induced a decrease in incorporated ³H-thymidine. The effect was noted even at the low antidepressant concentration of 0.5-1 μM. A more pronounced effect of inhibition was observed in the mitogen-induced proliferation (as reflected by the finding that at 10 μM, the level of proliferation of basal and stimulated cells was identical).

This data implicates a direct antiproliferative effect on the immune system induced by the antidepressants, which is especially evident when cells are exposed to activating agents.

### Example 4

### Effects of SSRIs Paroxetine and Sertraline on Concavalin A (Con-A)-Induced Mouse Splenocyte Proliferation

The effect of several psychotropic drugs on the proliferation of Con-A-induced mouse splenocyte proliferation was examined. The specific drugs evaluated included paroxetine and sertraline.

In particular, mouse splenocytes were isolated from C57BL healthy female mice. Cells (10,000/well) were then exposed to Con-A (5 μM) and treated with either the vehicle, with dexamethasone or with a drug (2.5-10 μM).

Cell viability was assessed 48 hr later using Alamar blue staining. The results are presented in FIG. 4, where each point is the graph represents the mean of 4 determinations. Results show that the SSRIs paroxetine and sertraline decreased cell viability to the same extent as compared to dexamethasone. Paroxetine and sertraline induce a dose-dependent decrease in mitogen-induced splenocyte mouse proliferation in a similar pattern to the corticosteroid dexamethasone. The IC50 determined for the antidepressant drugs was in the range of 2.5-5.5 μM.

### Example 5

### Effect of the SSRIs Paroxetine, Sertraline, Fluvoxamine and Venlafaxine on the Proinflammatory Cytokine INF-γ Secreted from Mouse Con-A Activated Splenocytes

INF-γ was determined in the supernatant of the splenocyte specimens using ELISA kit and protocol (Manufactured by Cytolab).

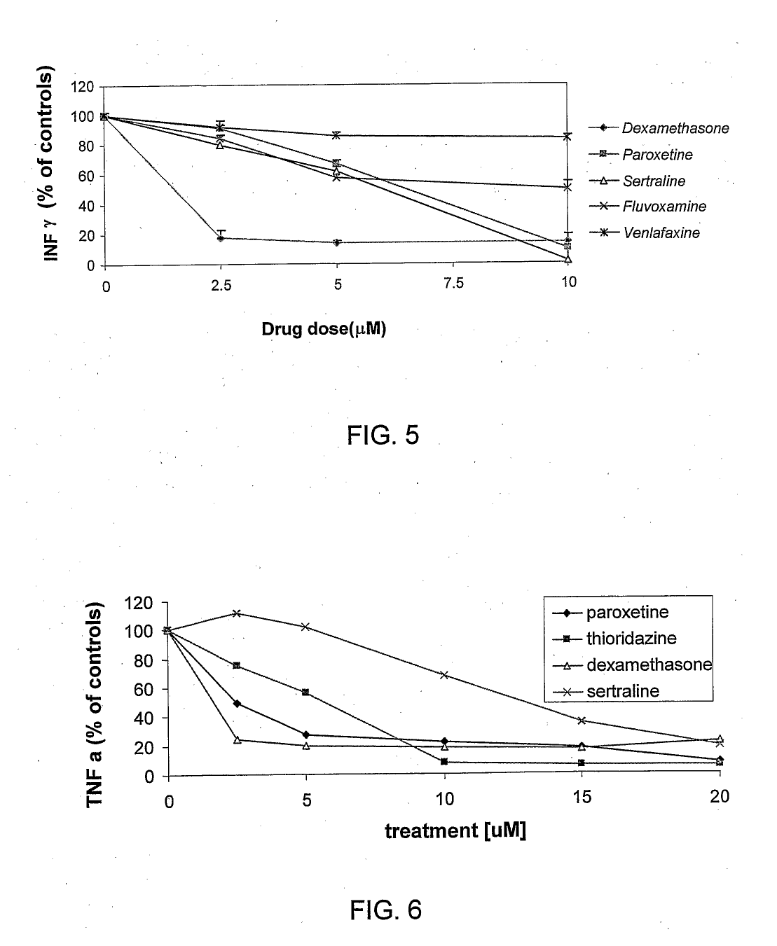

The results shown in FIG. 5 demonstrate that all agents induced a dose dependent decrease in INF-γ with dexamethasone showing the most dramatic effect (inhibition of 83% obtained at 2.5 μM), while sertraline and paroxetine induced similar or greater inhibition at the dose of 1 μM. Fluvoxamine and venlafaxine showed a lower effect reaching maximal inhibition of 51% and 16% at 10 μM respectively.

The data further shows that different SSRIs exert inhibition of inflammatory cytokine resembling the effect of glucocorticosteroids, with differential potency. Sertraline and paroxetine seem to have optimal activity.

### Example 6

### Effects of Paroxetine, Thioridazine and Sertraline on Con-A-Induced TNF-α Secretion

The effect of paroxetine, sertraline, and thioridazine on Con-A-induced TNF-α secretion in comparison with dexamethasone (all at concentrations of 2.5 20 μM) was also determined. Cells treated with Con-A alone served as the control (“Con-A”).

In particular, mouse splenocyte cells 1×10⁶ (cells/well) were exposed, for 48 hours, to the different drugs and Con-A and at the end of the experiment plates were centrifuged and supernatant collected for cytokine determination and TNF-α levels were determined in soups by ELISA (R&D # DY410 Minneapolis USA). Values are expressed in percent out of control (Vehicle-Con-A treated cells), data represent mean+SE of three experiments.

The sensitivity of the assay was 32 pg/ml, basal levels were below the assay sensitivity, and the Con-A induced TNF-α stimulation levels were 1067 pg/ml. The results shown in FIG. 6 indicate that the three SSRIs had a TNF-decreasing effect similar to dexamethasone.

### Example 7

### Effect of the SSRI Citalopram and the TCAs Amitriptyline and Doxepine on Mouse Con-A Induced Splenocyte Proliferation Using the Alamar Blue Test for Cell Viability

Cells were treated with vehicle, or the antidepressants (2.5-20 μM). Viability was assessed 48 hours later using alamar blue staining. (Each point is the mean+/−SEM of 4 determinations).

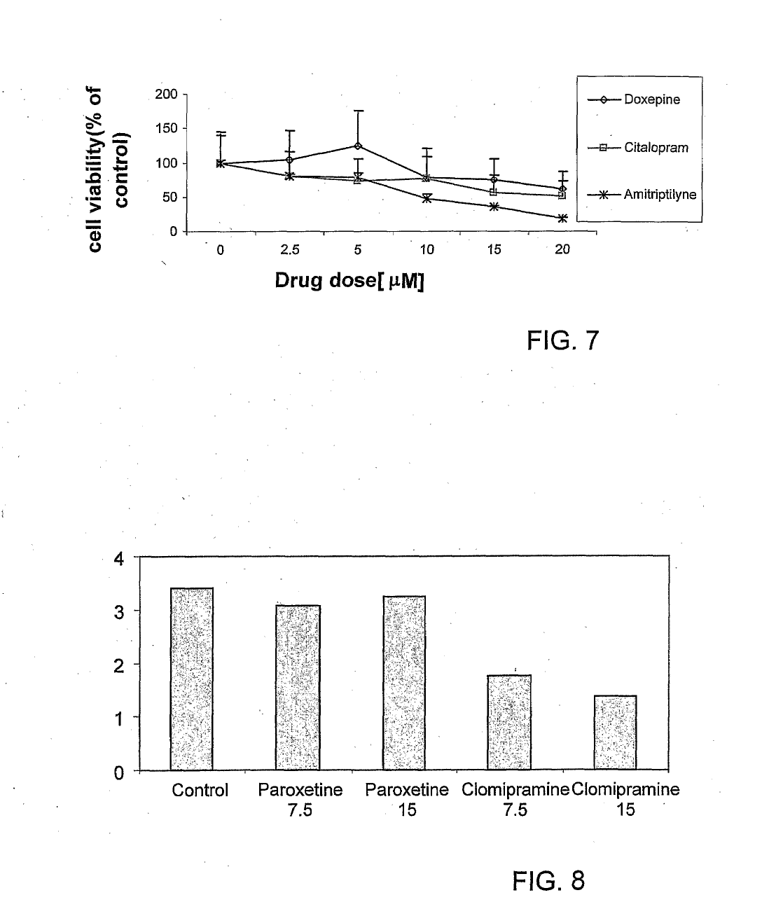

The data shown in FIG. 7 demonstrates that all agents induced a dose dependent decease in cell viability with amitriptyline presenting the highest activity (maximal inhibition of 80.7%, and citalopram and doxepine inducing 49% and 38% inhibition at 20 μM, respectively.

The data demonstrates the potential anti-proliferative and anti-inflammatory effect of additional agents from the SSRIs and TCA family. Amitriptyline seems to have the optimal effect among the tested agents.

### Example 8

### Effect of In Vivo 7 Day Treatment with Paroxetine or Clomipramine in C57 Black Male Mice, on the ConA-Induced Splenocyte Proliferation In Vitro (Con A at 5 μM)

Paroxetine or clomipramine were administered at 7.5 and 15 mg/kg x3/week i.p, respectively. Splenocytes were derived from treated animals.

Each group represents the mean of a pool of 5 animals. The results represented in FIG. 8 show that splenocytes derived from mice treated with clomipramine (TCA) but not with paroxetine, had lower capacity to respond to exogenous stimulus of Con-A, thus the ratio of Cona/basal was lower as compared to vehicle treated mice (control).

The data suggests that TCAs could have potential immunosuppressive effects, thus being efficacious in states of hyperactivated immune system.

### Example 9

### Effect of Paroxetine on Con-A-Induced Human Lymphocyte Proliferation

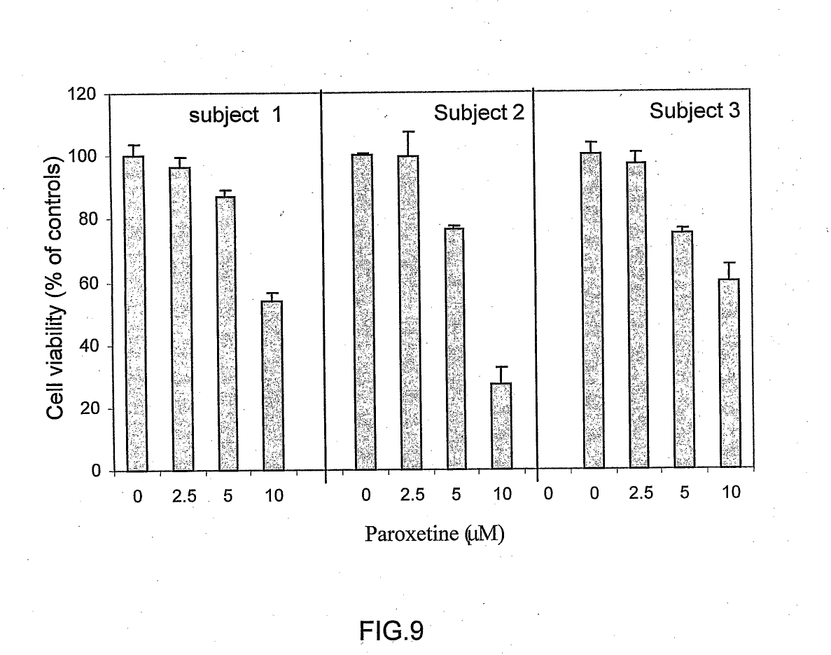

Human blood mononuclear cells (PBMC) were isolated from whole blood of 3 healthy volunteers using Ficoll-Paque gradient (Amersham Sweden). Lymphocyte proliferation was induced by exposure of 1×10⁶ cells/well to phytohemagglutinin (PHA) (2.5 μg/ml). Cells were exposed to PHA and treated with vehicle or with paroxetine (2.5-10.0 μM). Cell viability was assessed 48 hours post exposure to paroxetine (2.5-10.0 μM) using Alamar blue reagent (Wildflower, Santa Fe, N. Mex., USA) according to Nociari et al (1998). The results presented in FIG. 9 clearly show that paroxetine was able to inhibit human lymphocyte-induced proliferation in a dose dependent manner with IC50 of 5-10 μM.

### Example 10

### Effect of Paroxetine, Sertaline and Trifluoperazine on Human PBMC

Human PBMC were isolated from whole blood of 2 healthy volunteers (A and B subjects).

Cells (2×10⁶/ml) were exposed to LPS (2 ug/well) and treated with either the vehicle, or with paroxetine, sertraline, clomipramine and trifluoperazine (2.5-30 μM).

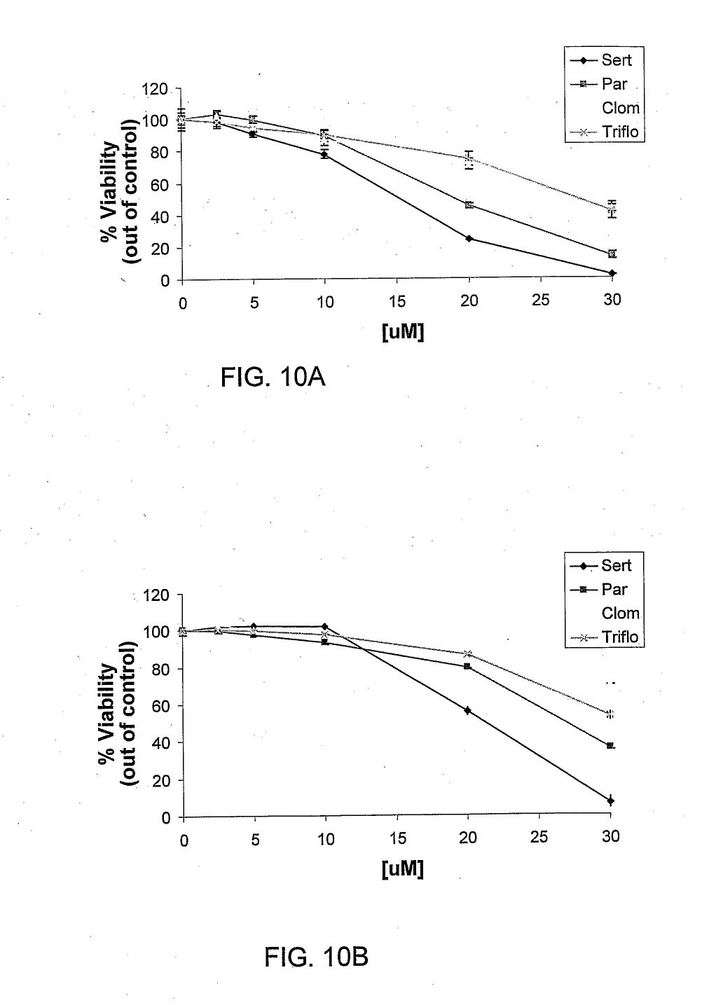

Cell viability was assessed 48 hours later using Alamar blue staining. The results are presented in FIGS. 10A and B, where each point in the graph represents the mean of 4 determinations. Results in both subjects show that all the agents induced a dose dependent decrease in cell viability with the following order of potency: sertraline>paroxetine>trifluoperazine>/=clomipramine.

The data shows that human immune cells (mainly lymphocytes) present high sensitivity to different psychotropics as reflected by attenuation of cell proliferation after exposure to SSRIs TCAs and phenothiazines.

### Example 11(A)

### Effect of Paroxetine, Sertraline and Dexamethasone on Expression of Cyclooxygenase-2 (Cox-2) in ConA Stimulated Mouse Splenocytes

Determination was performed using Western blot analysis and quantitative dencitometry of blot.

For each sample 2×10⁷ cells were used. Cells were lysed, and equal amounts of protein was fractionated on polyacrylamide gel and then transferred to PVDF membrane. The amount of the protein was detected by immuno-blotting with monoclonal anti-Cox-2 anti body (Murine anti COX2 Polyclonal Cat. 160126, Ann Arbor, Mich., USA. 2nd antibody conjugated to horseradish peroxidase was used and visualized with chemiluminescent Kit (Pierce, Rockford, Ill., USA) on film. Quantification of blot was performed using a densitometry apparatus (VersaDoc™ imaging system, BioRad, USA) and levels were normalized to actin.

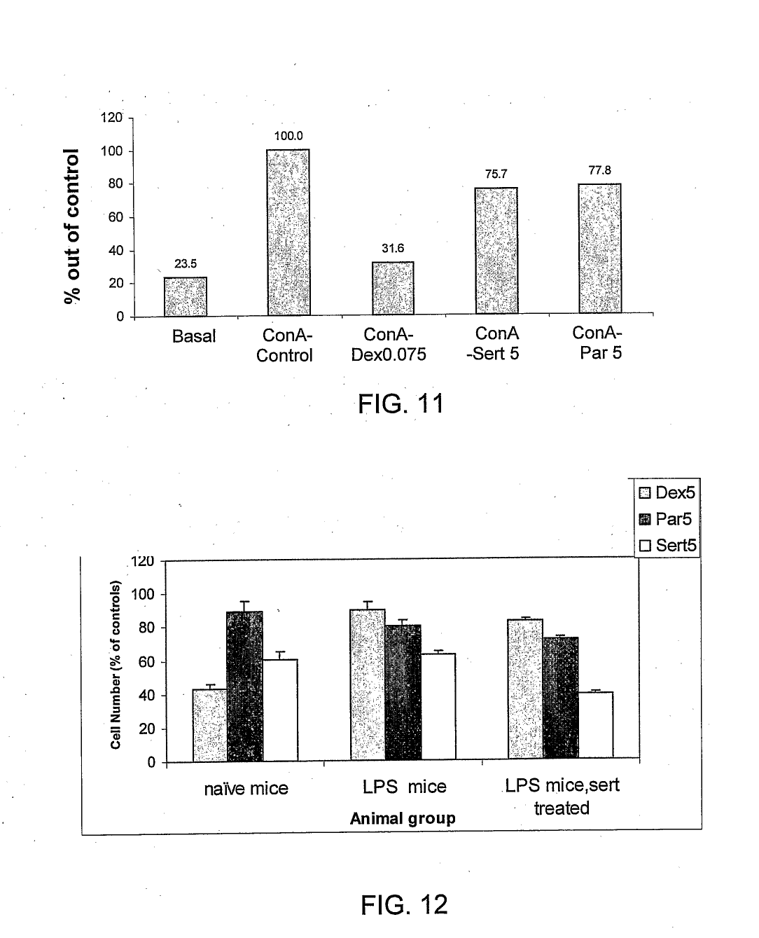

The data shows that Con-A markedly stimulated Cox-2 expression in mouse splenocytes (FIG. 11). This effect was completely antagonized by dexamethasone (0.075 μM). Surprisingly, it was found that also sertraline and paroxetine at 5 μM reduced Cox-2 expression by 24.3% and 22.2%, respectively.

This data point to the possibility that selected antidepressants such as SSRIs reduced Cox-2 expression, thus resulting in reduced secretion of the Cox-2 inflammatory products, i.e., prostaglandins.

### Example 11(B)

### Effect of Paroxetine on Stat3 and Cox-2 Expression in Human PHA Activated Lymphocytes

Total protein lysates from human lymphocyte culture were prepared by agitation with lysis buffer (20 mM Tris pH 7.5, 150 mM NaCl, 1 mM EDTA, 1% Nonidet P-40, 0.5% sodium deoxycholate, and 2 mM Na2VO4) for 1 h at 4° C. The proteins were cleared by centrifugation at 14,000×g for 20 min at 4° C. Protein concentration was determined using BCA kit (Pierce, Ill., USA). For Western blot analysis, 30 μg-protein from each sample were loaded in 7.5% SDS-PAGE. Electroblotted proteins were detected using specific anti COX-2 polyclonal antibody (Cayman chemical company, Michigan, USA) or an ti-Stat3 monoclonal antibody (Transduction Laboratories Lexington UK). Bound antibodies were visualized using chemiluminescence reaction.

Western blot analysis of Cox-2 and STAT3 in human lymphocytes showed that PHA 15 μM caused an increase in the expression of both Cox-2 and Stat3, and paroxetine at 5 μM, similarly to dexamethasone, antagonized the PHA-induced increase in both Stat3 and Cox-2 expression.

This data points to the possibility that selected antidepressants such as SSRIs reduce Cox-2 and STAT3 expression, thus resulting in reduced secretion of inflammatory products, i.e., prostaglandins.

### Example 12

### Comparative Effect of Paroxetine, Sertraline and Dexamethasone (5 μM each) on Viability of Con-A Activated Splenocytes (Pool of 5 Animals) Derived from Either Untreated (“naïve”) Mice, or from Lypopolysaccharide (LPS)-Treated Mice (as a Model of Inflammation)

LPS (manufactured by Sigma St. Louis Mo., USA, cat no L2880) was administered at 100 μg/mouse×2, on day 7 and day 14).

These effects are compared with LPS mice treated in vivo with sertraline (5 mg/kg×3/week for two weeks).

The results shown in FIG. 12 indicate that splenocytes from naïve animals responded most dramatically to dexamethasone (60% inhibition). Paroxetine had a slight effect of 10% and sertraline of 40%. LPS mice presented resistance to dexamethasone therapy and responded to paroxetine and sertraline in a manner similar to the naive animals, however LPS mice treated in vivo with sertraline were more responsive to either paroxetine or sertraline.

The data suggests that in an animal model of inflammation paroxetine and sertraline are effective in inhibiting the overwhelmed immune response even in states of resistance to glucocorticosteroids. Moreover it seems that in vivo therapy with an antidepressant e.g., sertraline, will not induce resistance, and can even sensitize the immune system to the therapeutic effect of the antidepressant.

### Example 13

### Effect of Psychotropic Agents on Human Colon Carcinoma Cells

Human Colon carcinoma (HT29) cell-line (ATCC USA) was maintained in RPMI medium supplemented with 10% fetal calf serum and penicillin (100 u/ml), streptomycin (100 mcg/ml), nystatin (12.5 u/ml), L-glutamine (2 mM). Confluent cultures were washed with phosphate-buffered saline (PBS), detached with trypsine (0.25%), centrifuged, and subcultured in 96-well microtiter test plates. Cells were exposed to vehicle or to psychotropic drugs. Cell viability was measured 24 hours later using the alamar blue and neutral red methods. Results were expressed as % of controls (vehicle treated cells). Each sample is the Mean+/−Sum of 4 determinations.

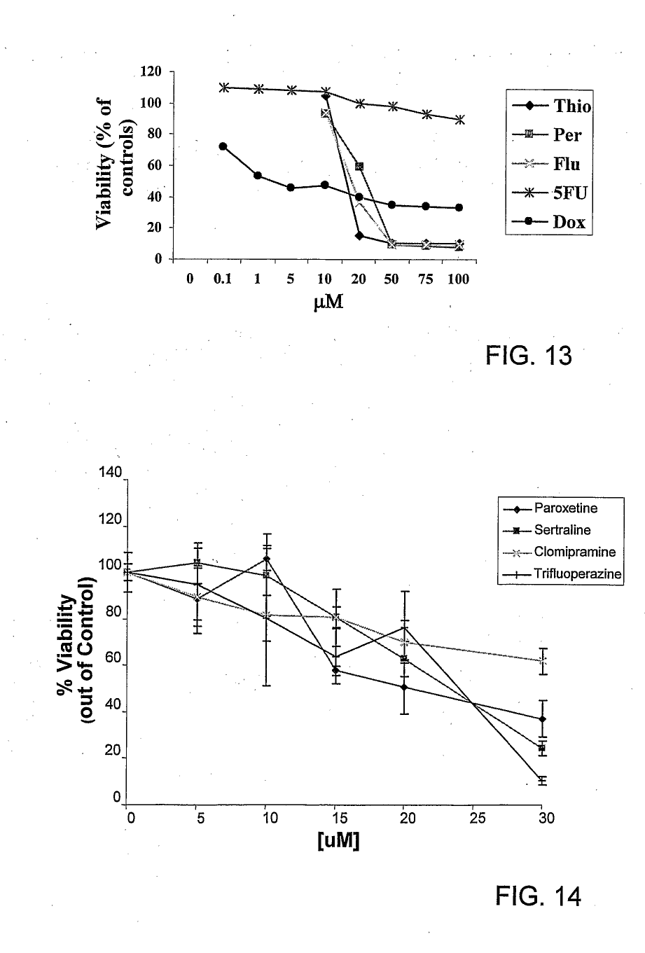

FIG. 13 shows that in HT29 cells the psychotropics thioridazine fluphenazine and perphenazine induced a dose dependent decrease in cell viability (thioridazine showed the highest activity 85% inhibition by 20 μM). The responsiveness to the psychotropics was found in the presence of resistance to the cytotoxic agent 5fluoro uracil (5FU), and only partial responsiveness to the cytotoxic agent doxorubicin.

FIG. 14 shows the effect of the SSRIs sertraline and paroxetine and the TCA clomipramine (10-30 μM) on the viability of human colon carcinoma (HT29) cell-line. All agents caused a dose dependent decrease in cell viability, with sertraline causing up to 75% inhibition, paroxetine 64% and clomipramine 38%. Each sample is the Mean+/−Sum of 4 determinations.

Collectively the data suggests that human GI tract cells are sensitive to antiproliferative effects of different psychotropics mainly phenothiazines, SSRIs and TCAs.

### Example 14

### Effect of Paroxetine and Sertraline on BCL-2 and on C-jun Expression in Human Colon Carcinoma HT29

The effect of paroxetine and sertraline on the expression of bcl-2 and C-jun was evaluated in human colon HT29 cell-line 24 hr after drug administration, using western blot analysis for C-jun and bcl-2 and specific antibodies.

The effect of paroxetine and sertraline 10 and 20 μM on the expression of the proteins bcl-2 and C-jun 24 hr after drug exposure is in the inhibition of the anti apoptotic protein bcl-2 and stimulation (10 and 20 μM) of the proapoptotic protein C-jun which is activated by JNK to execute apoptosis.

Collectively the data suggests that human GI tract cells are sensitive to antiproliferative and proapoptotic effects of different psychotropics mainly phenothiazines, SSRIs and TCAs.

### Example 15

### Anti-IBD Effect of Repeated Oral Administration of Paroxetine in a Rat Model of DSS-Induced Acute Colitis

Potential anti-IBD effect of two different doses of paroxetine, namely 0.2% and 0.4% was assessed in the rat model of DSS-induced acute colitis (DSS—dextran sulfate sodium). The use of DSS is a well-defined method for the induction of colitis' in animals. DSS can be controlled more precisely regarding molecular weight distribution, grade of sulfate incorporation and impurities than other agents typically used to achieve the same clinical end-point. This reagent model is suitable for screening of new drugs as it uses small animals, the oral administration of the agent is convenient to use in large groups of animals and the induced colitis responds to treatment with conventional drugs (see for example Axelsson et al, 1993).

Paroxetine (oral suspension, protected from light at or below 25° C.) was administered at two respective doses of 7.5 and 15 mg/kg by one daily repeated oral gavage (PO) for 16 days. Colitis induction commenced two days following initiation of treatment by continuous exposure ad libitum of the test animals to 3% DSS (36-50 kDa) in their drinking water (chlorinated and acidified, pH range of 5.1-5.9, freshly prepared on even test days). One test group subjected to identical dosing regimen conditions and DSS exposure was administered drinking water PO and another identically treated group but unexposed to DSS served for comparable assessment (water-treated non-DSS induced). Two additional paroxetine-treated non-DSS induced groups were likewise treated with the same selected does to detect any unforeseen paroxetine related effects. Test groups comprised either n=8 (DSS induced) or n=4 (non-DSS induced) male Sprague Dawley (SD) rats (healthy, young 0.8-9 weeks of age, total of 36 animals).

Evaluation of anti-IBD effect was based on the following four parameters: a. Daily clinical scoring of the characteristic experimental colitis signs, consisting of body weight loss, altered fecal consistency and hematochezia and expressed as Disease Activity Index (DAI); b. Body weight determination; c. Colon length; d. Macroscopic colon lesions scores noted at necropsy; and e. microscopic colon lesions score.

The histological scoring system for the 3% DSS-induced colonic inflammation is shown below in Tables 1 and 2:

TABLE 1
scoring system for the distribution of
lesions, reflecting lateral extension

| | |
|---|---|
| 0- | None (or 0%) |
| 1- | Focal (or 1-25% of the circumference of the intestinal section) |
| 2- | Multifocal (or 26-50% of the circumference of the intestinal section) |
| 3- | Nearly Diffuse (or 51-75% of the circumference of the intestinal |
| | section) |
| 4- | Diffuse (or 76-100% of the circumference of the intestinal section) |

TABLE 2
scoring system for the layers involved,
reflecting in-depth extension

0- No necrosis and/or ulceration
1- Half (up to 50%) mucosal necrosis and/or ulceration
2- Total (up to 100%) mucosal necrosis and/or ulceration
3- Mucosal and submucosal necrosis and/or ulceration
4- Transmural necrosis and/or ulceration

Final data evaluation was carried out by statistical analysis of differences between mean group values for each of the above listed parameters in the DSS-induced groups vs. respective mean values in the water-treated non-DSS induced group.

Mortality was confined to a total of 3 animals; two from the control DSS-induced group and one animal from the dose paroxetine-treated DSS-induced group. Onset was oh days 12, 13 and 10, respectively, with preceding overt disease-related clinical signs. No mortality occurred in any of the animals from the high-dose paroxetine-treated DSS-induced group and in any of the animals from the non-DSS groups.

Mean value of body weight (241-276 grams at study initiation, weight of animals at the time of treatment initiation did not exceed +/−20% of the mean weight of 255 grains) in the water-treated DSS-induced group revealed reduced gain from day 3 (p<0.05) and progressive decline from day 6 until study termination (p<0.01 days 7-10; p<0.05 days 11-13), as compared to control non-DSS group. Although a similar trend in mean body weight values was also noted in the paroxetine-treated DSS-induced groups, values did not reach statistical significance differences vs the respective control. Regardless of the treatment administered (water or paroxetine), all non-DSS induced groups, demonstrated similar progressive body weight gain throughout the entire study period.

Characteristic disease-related clinical signs were first noted in all DSS-induced groups at 24 hours following first exposure (day 1) and thereupon progressively developed in all DSS-induced animals (100% incidence of disease). Mean group values of DAI in the water-treated DSS-induced group steadily increased during exposure period, amounting to DAI=8 (maximum DAI=12) on study day 7 onwards, and were statistically significant vs the respective control group (p<0.01 on day 4, p<0.05 on day 6, 9-11, and p<0.01 on days 7-8 and 12-13). Mean group DAI values of both paroxetine-treated DSS-induced groups resembled those of the water-treated DSS-induced group, although the formers peak score was only noted on day 10 and significant differences vs control were of lower incidence (total of 5 days in the low paroxetine dose and total of 6 days in the high paroxetine dose, as opposed to a total of 9 days in the water-treated DSS-induced group).

Mean colon length in all DSS-induced groups was significantly shorter vs that in water-treated non-DSS induced group (p<0.01 water treated DSS-induced group; p<0.05 both paroxetine-treated DSS-induced groups).

Mean macroscopic colon scores for both the water-treated DSS-induced and the low dose paroxetine-treated DSS-induces groups were significantly higher vs control (p<0.01), whereas that of the high dose paroxetine-treated DSS-induced group did not significantly differ from control. Mean colon score of the high dose paroxetine-treated DSS-induced group was lower than that of the respective low dose paroxetine-treated group, suggesting also to a possible dose-response.

Statistical analysis of histopathological findings from all DSS-induced groups revealed that the medians of both scores (lateral and in-depth extensions of colon lesions) were significantly greater than zero. The medians of both paroxetine groups were lower than for the control group, but the differences did not reach statistical significance.

Under the experimental design conditions employed in this model it may be concluded that the paroxetine oral suspension reveal a trend of anti-IBD effect with a dose-response. Colon targeting is believed to decrease significantly the systemic level of paroxetine. The AUC value of paroxetine following administration of colon-targeted drug should be significantly lower due to the following reasons: when paroxetine is administered orally in suspension or tablets it is rapidly absorbed from the small intestine to the systemic circulation and its plasma concentration increases.

In contrast, after oral administration of the colon-targeted drug, only limited amount of the paroxetine is absorbed from the small intestine and most of the drug is localized in the large intestine (colon).

The same concept had been demonstrated with mesalamine using chitosan capsules wherein 50% decrease in systemic exposure was exhibited with colon targeting technology (see example 15).

### Example 16

### Chitosan Capsules Containing Paroxetine in DSS-Induced Colitis in Rats

As disclosed hereinbefore, such chitosan capsules are known to disintegrate in the colon, thereafter releasing their contents. Such capsules may be used for the local delivery of paroxetine hydrochloride or any other SSRI or TCA to the colon. Chitosan capsules (3.5 mm×1.6 mm) having a surface coated with for example hydroxypropyl methylcellulose phthalate as an enteric coating material may be prepared in accordance with known protocols (see for example Tozaki et al, 1997, 2002).

### Example 17

### Effect of Local Subcutaneous Administration of Sertraline on HT29 Human Colon Cancer Tumor Growth

The in-vivo anti tumor activity of active antidepressants was assessed in animal model of cancer. Five Nude mice (males, 6 weeks of age) were inoculated with HT29 cells (4,000,000/100 μL) by subcutaneous (s.c.) administration. One week after cell inoculation a clear solid tumor of small size (0.1-0.2 cm) appeared in 4 of the 5 mice. In the fifth mouse a bigger tumor (0.9 cm diameter) was observed. Sertraline therapy was initiated on the 7th day after cell grafting. 3 animals were treated with sertraline (20 mg/kg s.c×3/week) near the cancerous area and 2 animals served as controls. After 3 courses of sertraline administration a prevention of tumor growth (1 animal) and a decrease in tumor growth in the 2 other animals as compared to untreated controls.

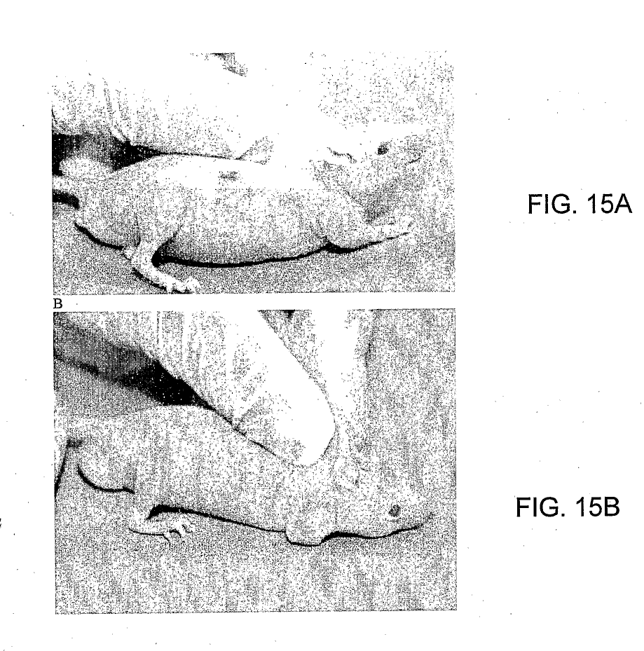

FIGS. 15A and B show a control vs sertraline treated mice 1 week after sertraline administration. These results clearly suggest that SSRIs such as sertraline and paroxetine may have an anti tumor effect against human colon cancer and that local or topical administration of the active antidepressants may be useful for the treatment of tumors and polyposes of the GI tract.

### REFERENCES

1. Axelsson et al, (1993) Inflammopharmacology, 2: 219-232.
2. Breese et al., (1994), Tumor necrosis factor producing cells in the intestinal mucosa of children with inflammatory bowel disease, Gastroenterology, 106:1455-1466.
3. Cianchi F. et al (2004), Cyclooxygenase-2 activation mediates the proangiogenic effect of nitric oxide in colorectal cancer. Clin. Cancer Res. 10(8):2694-704.
4. Giller et al., (1980), Am. J. Gastroenterol, 73:232-237.
5. Hu G H, et al (2005), Effect of normothermic liver ischemic preconditioning on the expression of apoptosis-regulating genes C-jun and Bcl-XL in rats. World J Gastroenterol, 11:2579-82
6. Moutaery A A (2005), Proglumide attenuates experimental colitis in rats; Experimental and Toxicologic Pathology, 56: 327-332.
7. Mudter J, et al (2005), Activation pattern of signal transducers and activators of transcription (STAT) factors in inflammatory bowel diseases. Am J Gastroenterol. 100:64-72.
8. Murch et al., (1991), Serum concentrations of tumour necrosis factor in childhood chronic inflammatory bowel disease, Gut. 32:913-917.
9. Nociari M M, Shalev A, Benias P, (1998), A novel one-step highly sensitive fluorimetric assay to evaluate cell-mediated cytotoxicity; J. Immunol. Methods, 213(2): 157-167.
10. Present et al., (1999), Infliximab for the treatment of fistulas in patients with Crohn's disease, N. Engl. J. Med. 340: 1398-1405.
11. Targan et al., (1997); A short-term study of chimeric monoclonal antibody cA2 to tumor necrosis factor for Crohn's disease: Crohn's Disease cA2 Study Group; N. Engl. J. Med. 337:1029-1035.
12. Talley, N. J., (2004), J. Am. J. Gastroenterol., 99(5):921-3.
13. Tozaki et al, (1997), Chitosan capsules for colon-specific drug delivery: improvement of insulin absorption from the rat colon; J. Pharm. Sci., 86:1016-1021.
14. Tozaki et al., (2002), Chitosan capsules for colon-specific drug delivery: enhanced localization of 5-aminosaluicyclic acid in the large intestine accelerates healing of TNBS-induced colitis in rats, J. Cont. Release, 82:51-61.

1. A method for the treatment of an Inflammatory Bowel Disease (IBD) or intestine polyposes in a subject comprising locally administering to the colon a therapeutically effective amount of at least one SSRI or at least one TCA.

2. The method according to claim 1, wherein said intestine polyposes are associated with IBD.

3. The method according to claim 1, wherein said intestine polyposes are not associated with IBD.

4. The method according to claim 1, wherein said IBD is selected from ulcerative colitis, callogenous colitis, microscopic colitis, lymphocytic colitis, ulcerative proctitis, rectocolitis, Crohn's disease, and infectious diarrhea.

5. The method according to claim 4, wherein said infectious diarrhea is associated with amebiasis, giardiasis, viral infection and pathogenic bacterial infection.

6. The method according to claim 1, wherein said at least one SSRI is selected from fluoxetine, norfluoxetine, paroxetine, duloxetine, tomoxetine (atomoxetine), nisoxetine, sertraline, desmethylsertraline, fluvoxamine, citalopram, apoxetine and venlafaxine.

7. The method according to claim 6, wherein said at least one SSRI is paroxetine.

8. The method according to claim 1, wherein said at least one TCA is selected from imipramine, clomipramine, amitriptyline and doxepine.

9. The method according to claim 8, wherein said at least one TCA is clomipramine.

10. The method according to claim 1, wherein said at least one SSRI or at least one TCA are administered orally.

11. The method according to claim 1, wherein said at least one SSRI or at least one TCA are administered rectally.

12. A pharmaceutical composition for the treatment of IBD or intestine polyposes comprising at least one SSRI or at least one TCA and a pharmaceutically acceptable carrier, excipient or diluent.

13. The composition according to claim 12, wherein said intestine polyposes are associated with IBD.

14. The composition according to claim 12, wherein said intestine polyposes are not associated with IBD.

15. The composition according to claim 12, wherein said IBD is selected from ulcerative colitis, callogenous colitis, microscopic colitis, lymphocytic colitis, ulcerative proctitis, rectocolitis, Crohn's disease, and infectious diarrhea.

16. The composition according to claim 15, wherein said infectious diarrhea is associated with amebiasis, giardiasis, viral infection and pathogenic bacterial infection.

17. The composition according to claim 12, wherein said at least one SSRI is selected from fluoxetine, norfluoxetine, paroxetine, duloxetine, tomoxetine (atomoxetine), nisoxetine, sertraline, desmethylsertraline, fluvoxamine, citalopram, apoxetine and venlafaxine.

18. The composition according to claim 17, wherein said at least one SSRI is paroxetine.

19. The composition according to claim 12, wherein said at least one TCA is selected from imipramine, clomipramine, amitriptyline and doxepine.

20. The composition according to claim 19, wherein said at least one TCA is clomipramine.

21. The composition according to claim 12 being suitable for oral administration.

22. The composition according to claim 12 being suitable for rectal administration.

23-33. (canceled)
